(19) United States
(12) Patent Application Publication
(10) Pub. No.: US 2017/0050897 A1
(43) Pub. Date: Feb. 23, 2017

(54) BENZENE PRODUCTION FROM ETHANOL OVER GOLD/TITANIUM DIOXIDE CATALYSTS

(71) Applicant: SABIC Global Technologies B.V., Bergen op Zoom (NL)

(72) Inventor: Hicham Idriss, Thuwal (SA)

(21) Appl. No.: 15/308,514

(22) PCT Filed: Jun. 22, 2015
(86) PCT No.: PCT/IB2015/054672

§ 371 Date: Nov. 2, 2016

Related U.S. Application Data
(60) Provisional application No. 62/015,687, filed on Jun. 23, 2014.

(51) Int. Cl. C07C 1/24; B01J 21/06; B01J 37/08; B01J 35/02; B01J 35/10; B01J 37/02; B01J 23/52; B01J 35/00

(52) U.S. Cl. CPC C07C 1/24; B01J 23/52; B01J 21/063; B01J 35/0013; B01J 35/026; B01J 35/023; B01J 35/1061; B01J 35/1057; B01J 37/0221; B01J 37/08

(57) ABSTRACT

Disclosed is a catalyst capable of producing benzene from ethanol comprising a titanium dioxide support, gold nanostructures dispersed on the surface of the titanium dioxide support, and ethanol adsorbed onto the surface of the titanium dioxide support, wherein the catalyst is capable of producing benzene from the adsorbed ethanol such that the benzene carbon yield from the adsorbed ethanol is at least 10% when the catalyst is heated to a temperature of 350 to 700 K.

### CROSS REFERENCE TO RELATED APPLICATIONS

This application claims benefit to U.S. Provisional Patent Application No. 62/015,687 titled “BENZENE PRODUCTION FROM ETHANOL OVER GOLD/TITANIUM DIOXIDE CATALYSTS” filed Jun. 23, 2014. The entire contents of the referenced patent are incorporated herein by reference.

### BACKGROUND OF THE INVENTION

A. Field of the Invention

The invention generally concerns catalysts and catalytic processes that can be used to produce benzene or hydrogen, or both, from ethanol. The catalysts include titanium dioxide as the catalytic support with metal particles dispersed on the surfaces of the titanium dioxide support. The titanium dioxide support can be nanostructures of rutile or anatase form, and the metal particles can be gold nanostructures.

B. Description of Related Art

Benzene is widely used in the chemical industry to produce several downstream products. For instance, benzene can be converted into ethyl benzene, which is used to produce styrene and ultimately polystyrene. Benzene is also widely used to produce cumene, which is used to produce phenol, a component in phenolic resins and adhesives. Additional chemical products obtained from benzene include cyclohexane (a precursor of caprolactam and adipic acid, both of which are found in nylon) and aniline (which is used to produce methylene diphenyl diisocyanate (MDI)). FIG. 1 provides an illustration of the various downstream chemicals obtained from benzene. Ultimately, these downstream chemicals find their way into a variety of commercial products such as clothing, paints, adhesives, windows, etc.

Similarly, hydrogen is widely sought for its high energy value as well as an important chemical compound needed for many processes such as ammonia synthesis.

While various chemical processes are used to produce benzene, catalytic hydrocracking of hydrocarbons over Pt or Ru based catalysts is the most prevalent process. This process relies on hydrocarbon feed, which is generally obtained from petroleum. Petroleum, however, is oftentimes characterized as a limited resource that requires a substantial amount of effort to obtain. This can increase the costs associated with benzene production.

Some attempts have even been made to produce benzene from ethanol via catalytic supports (see Idriss et al. Platinum Metals Review. 2004, 48(3):105-115). However, such attempts have proven to be inefficient in that the benzene carbon yield from ethanol can be below 10% or can have several complicated processing steps or both.

### SUMMARY OF THE INVENTION

A solution to the aforementioned inefficiencies surrounding benzene production has been discovered. The solution resides in obtaining benzene from ethanol at high yields and from ethanol feeds rather than from hydrocarbon feeds. Most notably, the processes of the present invention provide for an efficient conversion process of ethanol to benzene (e.g., benzene carbon yield from ethanol can be up to 70%), thereby providing for a commercially viable benzene production process. The process of the present invention can also produce hydrogen gas (H₂). The high conversion rate is due to the gold/titanium dioxide catalysts of the present invention. In particular, gold nanostructures are supported by titanium dioxide, which allows for efficient adsorption of ethanol onto the catalyst and subsequent conversion of ethanol to benzene. Without wishing to be bound by theory, it is believed that the catalysts of the present invention shift the reaction selectivity from ethylene production to benzene production via increased conversion of the adsorbed ethanol to acetaldehyde (via dehydrogenation) rather than to ethylene (via dehydration). Ethylene, once formed, desorbs quickly from the catalytic surface. By comparison, however, acetaldehyde has a stronger adsorption profile than ethylene, thereby allowing it to further react with the catalytic surface in a series of condensation reactions (e.g., beta-aldolization) to crotonaldehyde (e.g., C₄ unsaturated aldehyde), which further reacts with another adsorbed acetaldehyde to produce a C₆ unsaturated aldehyde. At the interface between the gold nanostructures and the titanium dioxide support the C₆ unsaturated aldehyde gives benzene with a cyclic reaction.

In one embodiment of the present invention there is disclosed a catalyst capable of producing benzene from ethanol. The catalyst can further produce hydrogen gas (H₂) along with benzene. The catalyst can include a titanium dioxide support, gold nanostructures dispersed on the surface of the titanium dioxide support, and ethanol adsorbed onto the surface of the titanium dioxide support. Such catalysts are capable of producing benzene from the adsorbed ethanol to the extent that the benzene carbon yield from the adsorbed ethanol is at least 10% when the catalyst is heated to a temperature of 350 to 700 Kelvin (K) (about 76 to 427° C.). The catalysts of the present invention can also have acetaldehyde or 2-4-hexadienal (or both) present on their surfaces. In certain aspects, the catalysts can have 1 to 10 wt. %, 2 to 10 wt. %, 3 to 10 wt. %, 4 to 10 wt. %, 5 to 10 wt. %, 6 to 10 wt. %, 7 to 10 wt. %, 8 to 10 wt. %, 9 to 10 wt. %, or can have more than 10 wt. % (e.g., 11, 12, 13, 14, 15, 20, 25, 30, 40, or 50 wt. % or more) of the gold nanostructures. In certain aspects, the titanium dioxide support comprises titanium dioxide nanostructures or microstructures. The gold nanostructures and the titanium dioxide nanostructures or microstructures can each individually be spherical in shape, elongated or rod or fiber-shaped, or have irregular shapes or other shapes, including those described below. Still further, the titanium dioxide support could be shaped to be film or sheet. In certain aspects, the nanostructures of the gold or titanium dioxide have an average size of 10 to 20 nm and the nano-fibers have an average width of 10 to 30 nm and an average length of 40 to 60 nm. In some aspects, the titanium dioxide support can have an inverse opal structure. The inverse opal structure can have pores having an average size of 175 to 400 nm or preferably from 175 to 250 nm. In some particular instances, the titanium dioxide can be reduced (e.g., by H₂ gas). In some aspects, the titanium dioxide support has an average pore size of less than 10 nm or less than 5 nm. In one instance, the gold nanostructures can be nanoparticles having an average size of less than 15 nm, less than 10 nm, or less than 5 nm. The catalysts of the present invention can be capable of producing benzene from the adsorbed ethanol such that the benzene carbon yield from ethanol is at least 10, 20, 30, 40, 50%, 60%, or 70%, or from 10 to 70%, 20 to 70%, 30 to 70%, 40 to 70%, 50 to 70%, 60 to 70%, or greater than 70% (e.g., 75, 80, 85, or 90% or more) when the catalyst is heated to a temperature of 350 to 700 K, 400 to 700 K, or 500 to 700 K. In a preferred aspect, the temperature range can be 550 to 650 K or 550 to 600 K or about 585 K and the majority of carbon from the adsorbed ethanol is present in the produced benzene. The catalysts of the present invention can be in particulate or powdered form or can take the form of a sheet or thin film.

Also disclosed is a method of producing benzene from any one of the catalysts of the present invention. Hydrogen gas (H₂) can also be produced along with benzene via the methods of the present invention. The method can include heating the catalyst to a temperature of 350 to 700 K, wherein benzene is produced such that the benzene carbon yield from ethanol is at least 10%. As noted above and throughout this specification, the benzene carbon yield from ethanol can be at least 10, 20, 30, 40, 50%, 60%, or 70%, or from 10 to 70%, 20 to 70%, 30 to 70%, 40 to 70%, 50 to 70%, 60 to 70%, or greater than 70% (e.g., 75, 80, 85, or 90% or more) when the catalyst is heated to a temperature of 350 to 700 K, 400 to 700 K, or 500 to 700 K. In a preferred aspect, the temperature range can be 550 to 650 K or 550 to 600 K or about 585 K and the majority of carbon from the adsorbed ethanol is present in the produced benzene. In some instances, the benzene carbon yield from the adsorbed ethanol can be from 5 to 15% at a temperature of 350 to 400 K and 40 to 60% at a temperature of 550 to 650 K. The benzene can be produced and desorbed at the interface of the titanium dioxide support and the gold nanostructures. In a particular embodiment, the benzene can be formed by cyclization of a hexadienal intermediate. The hexadienal intermediate can be derived from the adsorbed ethanol. In one instance of the present invention, the adsorbed ethanol can be bioethanol (ethanol derived from biomass). The produced benzene can be collected and stored and/or further processed into other chemicals including those described in FIG. 1 (non-limiting examples of such chemicals that can be produced from benzene include styrene, cumene, cyclohexane, aniline, or a chlorobenzene).

In another aspect of the present invention there is disclosed a method of producing any one of the catalysts of the present invention. The method can include: (a) dispersing gold nanostructures onto at least a portion of a surface of a titanium dioxide support to produce titanium dioxide supported gold nanostructures; (b) contacting the titanium dioxide supported gold nanostructures with ethanol under conditions sufficient to adsorb ethanol onto the surface of the titanium dioxide; and (c) obtaining the catalyst of the present invention. The titanium dioxide supported gold nanostructures from step (a) can be reduced with a reducing agent (e.g., H₂ gas) prior to step (b) or after step (b) or both before and after step (b). The temperature at which the reducing step can take place can vary as desired. In some preferred aspects, the reducing temperature can be 200° C. to 500° C. In other instances, step (b) can be performed at a temperature of 150° C. to 500° C. Still further, the ethanol from step (b) can be comprised within a feed stream that includes ethanol or bioethanol or both. In preferred aspects, the produced catalysts of the present invention can have up to 10 wt. % of the gold nanostructures. However, and as explained above and throughout this specification, the produced catalysts can have 1 to 10 wt. %, 2 to 10 wt. %, 3 to 10 wt. %, 4 to 10 wt. %, 5 to 10 wt. %, 6 to 10 wt. %, 7 to 10 wt. %, 8 to 10 wt. %, 9 to 10 wt. %, or can have more than 10 wt. % (e.g., 11, 12, 13, 14, 15, 20, 25, 30, 40, or 50 wt. % or more) of the gold nanostructures. Still further, the amount of gold nanostructure loading, the size of the gold nanostructures, the shapes of the gold nanostructures, the size of the titanium dioxide supports, the shapes of the titanium oxide supports, and the various titanium dioxide phases (e.g., anatase, rutile, and brookite) can be varied as desired to achieve a particular result.

In the context of the present invention, embodiments 1-30 are described. Embodiment 1 is a catalyst capable of producing benzene from ethanol that can include: a titanium dioxide support; gold nanostructures dispersed on the surface of the titanium dioxide support; and ethanol adsorbed onto the surface of the titanium dioxide support. The catalyst can be capable of producing benzene and hydrogen from the adsorbed ethanol such that the benzene carbon yield from the adsorbed ethanol is at least 10% when the catalyst is heated to a temperature of 350 to 700 K. Embodiment 2 is the catalyst of embodiment 1, wherein the catalyst include 1 to 10 wt. % or 4 to 8 wt. % or 6 to 8 wt. % of the gold nanostructures. Embodiment 3 is the catalyst of any one of embodiments 1 to 2, wherein the titanium dioxide support includes titanium dioxide nanostructures or microstructures. Embodiment 4 is the catalyst of embodiment 3, wherein the titanium dioxide nanostructures include nanoparticles or nano-fibers or a combination thereof. Embodiment 5 is the catalyst of embodiment 4, wherein the nanoparticles have an average size of 10 to 20 nm and the nano-fibers have an average width of 10 to 30 nm and an average length of 40 to 60 nm. Embodiment 6 is the catalyst of any one of embodiments 1 to 5, wherein the titanium dioxide support has an inverse opal structure. Embodiment 7 is the catalyst of embodiment 6, wherein the inverse opal structure has pores having an average size of 175 to 400 nm. Embodiment 8 is the catalyst of any one of embodiments 1 to 7, wherein the titanium dioxide is reduced titanium dioxide. Embodiment 9 is the catalyst of any one of embodiments 1 to 8, wherein the titanium dioxide has an average pore size of less than 10 nm or less than 5 nm. Embodiment 10 is the catalyst of any one of embodiments 1 to 9, wherein the gold nanostructures are nanoparticles having an average size of less than 15 nm, less than 10 nm, or less than 5 nm. Embodiment 11 is the catalyst of any one of embodiments 1 to 10, wherein the catalyst is capable of producing benzene from the adsorbed ethanol such that the benzene carbon yield from ethanol is at least 20, 30, 40, 50%, or 60% when the catalyst is heated to a temperature of 500 to 700 K. Embodiment 12 is the catalyst of any one of embodiments 1 to 10, wherein the catalyst is capable of producing benzene from the adsorbed ethanol such that the benzene carbon yield from ethanol is 10 to 70% or 20 to 70% or 30 to 70% or 40 to 70% when the catalyst is heated to a temperature of 500 to 700 K. Embodiment 13 is the catalyst of embodiment 12, wherein the temperature is 550 to 650 K or 550 to 600 K or about 585 K and the majority of carbon from the adsorbed ethanol is present in the produced benzene. Embodiment 14 is the catalyst of any one of embodiments 1 to 13, wherein the catalyst is in particulate or powdered form.

Embodiment 15 is a method of producing benzene from any one of the catalysts of embodiments 1 to 14. The method can include heating any one of the catalysts of embodiments 1 to 14 to a temperature of 350 to 700 K, wherein benzene is produced such that the benzene carbon yield from ethanol is at least 10%. Embodiment 16 is the method of embodiment 15, wherein the benzene carbon yield from ethanol is at least 10% up to 70%. Embodiment 17 is the method of any one of embodiments 15 to 16, wherein the benzene carbon yield from the adsorbed ethanol is 5 to 15% at a temperature of 350 to 400 K and 40 to 60% at a temperature of 550 to 650 K. Embodiment 18 is the method of any one of embodiments 15 to 17, wherein benzene is produced and desorbed at the interface of the titanium dioxide support and the gold nanostructures. Embodiment 19 is the method of embodiment 18, wherein the benzene is formed by cyclization of a hexadienal intermediate. Embodiment 20 is the method of any one of embodiments 15 to 19, wherein the adsorbed ethanol is bioethanol. Embodiment 21 is the method of any one of embodiments 15 to 20, wherein the produced benzene is collected and stored. Embodiment 22 is the method of any one of embodiments 15 to 21, wherein the produced benzene is used to produce a compound. Embodiment 23 is the method of embodiment 22, wherein the compound is styrene, cumene, cyclohexane, aniline, or a chlorobenzene.

Embodiment 24 is a method of producing any one of the catalysts of embodiments 1 to 14, the method can include: (a) dispersing gold nanostructures onto at least a portion of a surface of a titanium dioxide support to produce titanium dioxide supported gold nanostructures; (b) contacting the titanium dioxide supported gold nanostructures with ethanol under conditions sufficient to adsorb ethanol onto the surface of the titanium dioxide; and (c) obtaining the catalyst. Embodiment 25 is the method of embodiment 24, wherein the titanium dioxide supported gold nanostructures from step (a) are reduced with a reducing agent prior to step (b). Embodiment 26 is the method of embodiment 25, wherein the reducing agent is hydrogen gas. Embodiment 27 is the method of any one of embodiments 25 to 26, wherein the titanium dioxide supported gold nanostructures are reduced at a temperature of 200° C. to 500° C. Embodiment 28 is the method of any one of embodiments 24 to 27, wherein step (b) is performed at a temperature of 150° C. to 500° C. Embodiment 29 is the method of any one of embodiments 24 to 28, wherein the ethanol from step (b) is included within a feed stream that includes bioethanol. Embodiment 30 is the method of any one of embodiments 24 to 29, wherein the produced catalyst can include 1 to 8 wt. % or 4 to 8 wt. % or 6 to 8 wt. % of the gold nanostructures.

“Nanostructure” refers to an object or material in which at least one dimension of the object or material is equal to or less than 100 nm (e.g., one dimension is 1 to 100 nm in size), unless otherwise indicated. In a particular aspect, the nanostructure includes at least two dimensions that are equal to or less than 100 nm (e.g., a first dimension is 1 to 100 nm in size and a second dimension is 1 to 100 nm in size). In another aspect, the nanostructure includes three dimensions that are equal to or less than 100 nm (e.g., a first dimension is 1 to 100 nm in size, a second dimension is 1 to 100 nm in size, and a third dimension is 1 to 100 nm in size). The shape of the nanostructure can be of a fiber, a wire, a particle, a sphere, a rod, a tetrapod, a hyper-branched structure, or mixtures thereof.

“Microstructure” refers to an object or material in which at least one dimension of the object or material is greater than 0.1 μm and up to 100 μm and in which no dimension of the object or material is 0.1 μm or smaller. In a particular aspect, the microstructure includes two dimensions that are greater than 0.1 μm and up to 100 μm (e.g., a first dimension is greater than 0.1 μm and up to 100 μm in size and a second dimension is greater than 0.1 μm and up to 100 μm in size). In another aspect, the microstructure includes three dimensions that are greater than 0.1 μm and up to 100 μm (e.g., a first dimension is greater than 0.1 μm and up to 100 μm in size, a second dimension is greater than 0.1 μm and up to 100 μm in size, and a third dimension is greater than 0.1 μm and up to 100 μm in size).

The terms “about” or “approximately” are defined as being close to as understood by one of ordinary skill in the art, and in one non-limiting embodiment the terms are defined to be within 10%, preferably within 5%, more preferably within 1%, and most preferably within 0.5%.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular components, compositions, ingredients, etc. disclosed throughout the specification. With respect to the transitional phase “consisting essentially of,” in one non-limiting aspect, a basic and novel characteristic of the catalysts and methods of the present invention are their ability to efficiently produce benzene from ethanol.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1: Downstream chemicals that can be produced from benzene.

FIGS. 2A and B: Illustrations of catalysts of the present invention.

FIG. 3: Illustration of a reactor comprising a catalyst of the present invention.

FIG. 4: XRD patterns for TiO₂ anatase nanoparticles (A) and rutile nanofibers (R) with different Au loadings, TiO₂ inverse opal (IO) and micron sized Au metal powder as indicated.

FIGS. 5A and 5B: Transmission electron microscopic (TEM) images of 8 wt % Au/TiO₂ anatase (A) and rutile (B). The rectangle in each main picture is magnified and presented at the top right corner to highlight the Au particles on the surface of the TiO₂ support.

FIGS. 6A to 6D: Scanning electron microscopic (SEM) images of TiO₂ inverse opal (FIG. 6A) and TEM images (FIGS. 6B-6D) of the similar area represented by the square in images a, b, and c, respectively, to demonstrate the porous structure and the size and dispersion of the Au particles.

FIG. 7: Temperature programmed desorption (“TPD”) profile of different desorption products after ethanol adsorption at room temperature on H₂-reduced anatase TiO₂ nanoparticles.

FIG. 8: TPD profile of different desorption products after ethanol adsorption at room temperature on H₂-reduced Au/TiO₂.

FIG. 9: Desorption profile of benzene from TPD of ethanol adsorbed on TiO₂ and Au/TiO₂ with indicated Au loading.

FIG. 10: TPD desorption profiles of ethylene after ethanol adsorption on Au/TiO₂ anatase catalyst as a function of Au loading.

FIG. 11: TPD desorption profiles of acetaldehyde after ethanol adsorption on Au/TiO₂ catalyst as a function of Au loading.

FIG. 12: TPD desorption profiles of hydrogen after ethanol adsorption on Au/TiO₂ rutile catalyst as a function of Au loading.

### DETAILED DESCRIPTION OF THE INVENTION

While plant-based fuels and feeds such as bioethanol are readily available, their use in downstream chemical processing is severely limited. Instead, the chemical industry relies heavily on fossil fuels. For instance, benzene production is primarily derived from petroleum.

The processes of the present invention provide for an alternative feed source for producing benzene or hydrogen, or both. In particular, ethanol (e.g., bio-based ethanol′) can be used to efficiently produce benzene and hydrogen via the catalysts and processes of the present invention. As illustrated in non-limiting embodiments of the present invention, the gold nanostructure/titanium dioxide catalysts of the present invention can be used to convert ethanol into benzene at commercially relevant yields (e.g., greater than 10% and up to 70% conversion yields). Stated plainly, the present invention offers a commercially viable benzene production process from a feed source that can be based on biofuels rather than fossil fuels.

These and other non-limiting aspects of the present invention are discussed in further detail in the following sections.

### A. Gold Nanostructure/Titanium Dioxide Catalysts

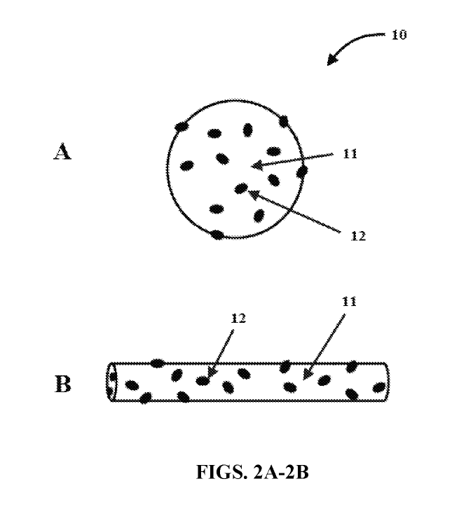

Referring to FIG. 2, the catalysts (10) of the present invention include titanium dioxide (11) and gold nanostructures (12) dispersed on the surface of the titanium dioxide (11). Titanium dioxide (11) can be in the form of anatase, rutile, or brookite phases, or combinations thereof. Anatase and rutile phases have a tetragonal crystal system, whereas the brookite phase has an orthorhombic crystal system. Each of the different phases can be purchased from various manufactures and supplies (e.g., Titanium (IV) oxide anatase Nano powder and Titanium (IV) oxide rutile Nano powder in a variety of sizes and shapes can be obtained from Sigma-Aldrich® Co. LLC (St. Louis, Mo., USA) and from Alfa Aesar GmbH & Co KG, A Johnson Matthey Company (Germany)); Enamel Grade Titanium dioxide (Brookite) from Yixing Zhenfen Medical Chemical Co., Ltd. (China); all phases of titanium dioxide from L.E.B. Enterprises, Inc. (Hollywood, Fla. USA)). Alternatively, the titanium dioxide 20 can be made by any process known by those of ordinary skill in the art (e.g., precipitation/co-precipitation, sol-gel, template/surface derivatized metal oxide synthesis, solid-state synthesis of mixed metal oxides, micro emulsion technique, solvothermal, sonochemical, combustion synthesis, etc.).

With respect to the gold nanostructures (12), such materials can be obtained from a variety of commercial sources in a variety of forms (e.g., particles, rods, films, etc.) and sizes (e.g., Nano scale or Micro scale). By way of example, each of Sigma-Aldrich® Co. LLC and Alfa Aesar GmbH & Co KG offer such products. Alternatively, they can be made by any process known by those of ordinary skill in the art. In a non-limiting aspect, the gold nanostructures (12) can be prepared using co-precipitation or deposition-precipitation methods (Yazid et al.). The gold nanostructures (12) can be substantially pure or can also be binary or tertiary alloys (e.g., Au+another metal—e.g., Pd, Ag, etc.). The gold nanostructures (12) can be of any size compatible with the titanium dioxide (11) support. In some embodiments, the gold nanostructures (12) are nanowires, nanoparticles, nanoclusters, nanocrystals, or combinations thereof.

The shape of the catalysts (10) can largely be controlled by the shape of the titanium dioxide support (11). By way of example only, the titanium dioxide support (11) can have a substantially spherical shape (FIG. 2A) or be substantially rod-shaped (FIG. 2B). Alternatively, the support (11) can have an irregularly shape (not shown) or can be formed into sheets or films (also not shown). Still further, the titanium dioxide support (11) can be a single phase such that it contains only anatase, rutile, or brookite, or can be mixed-phase such that it contains both anatase and rutile phases or anatase, rutile, and brookite phases.

The catalysts (10) of the present invention can be prepared from the aforementioned titanium dioxide material (11) and the gold nanostructures (12) by using the process described in the Examples section of this specification. Other optional methods that can be used to make the catalysts (10) of the present invention include formation of aqueous solutions of titanium dioxide ions in the presence of gold nanostructures (12) followed by precipitation, where the gold nanostructures (12) are attached to at least a portion of the surface of precipitated titanium dioxide crystals or particles or rods (10). Alternatively, the gold nanostructures (12) can be deposited on the surface of the titanium dioxide crystals, particles, or rods (11) by any process known by those of ordinary skill in the art. Deposition can include attachment, dispersion, and/or distribution of the gold nanostructures (12) on the surface of the titanium dioxide (11). As another non-limiting example, the titanium dioxide (11) material can be mixed in a volatile solvent with the gold nanostructures (12). Another example includes deposition precipitation whereby gold ions soluble in acidic solution are predicated over TiO₂ using urea (See, Photonic Band Gap Au/TiO₂ materials as highly active and stable Photocatalysts for Hydrogen production from water by Waterhouse et. al, Scientific Reports, 3, 2849 (1-5) (2013)). After stirring and sonication, the solvent can be evaporated off. The dry material can then be ground into a fine powder and calcined (such as at 300° C.) to produce a catalysts (10) of the present invention. Calcination (such as at 300° C.) can be used to further crystalize the titanium dioxide support (11).

### B. Catalytic Reactor Systems

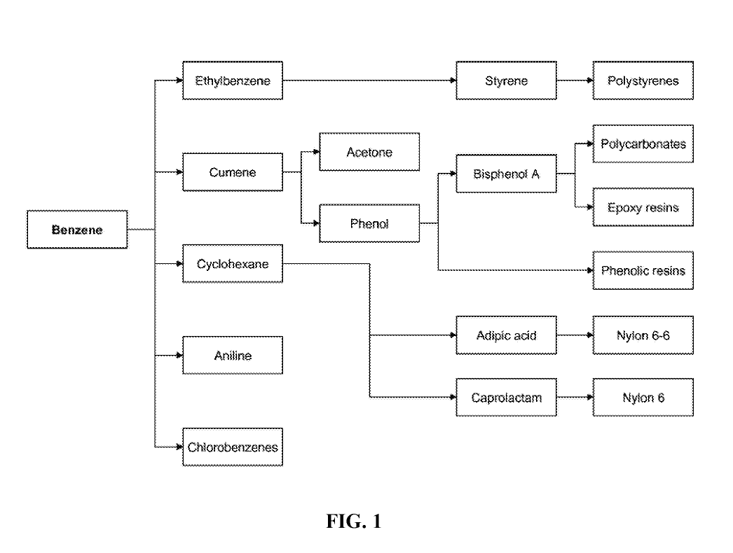

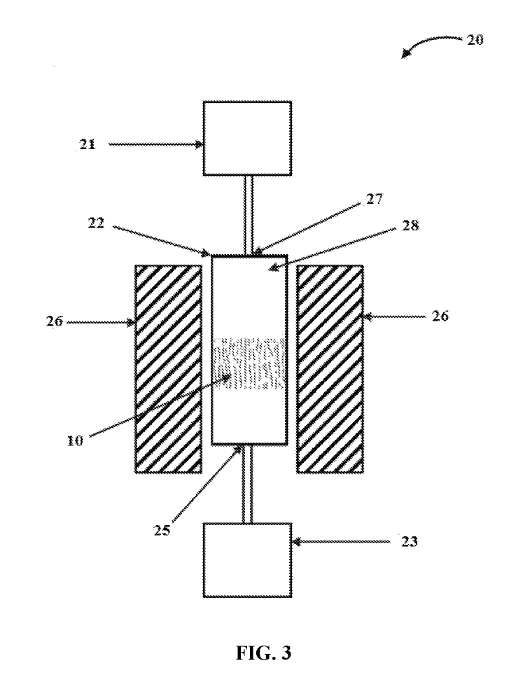

Referring to FIG. 3, a system (20) is illustrated, which can be used to convert ethanol to benzene with the catalysts (10) of the present invention. The system (20) can include an ethanol source (21), a reactor (22), and a collection device (23). The ethanol source (21) can be configured to be in fluid communication with the reactor (22) via an inlet (27) on the reactor (22). The ethanol source (21) can be configured such that it regulates the amount of ethanol feed entering the reactor (22). The reactor (22) can include a reaction zone (28) having the catalyst (10) of the present invention. The amount of the catalyst (10) used can be modified as desired to achieve a given amount of product produced by the system (20). A non-limiting example of a reactor (22) that can be used is a fixed-bed reactor (e.g., a fixed-bed tubular quartz reactor which can be operated at atmospheric pressure). The reactor (22) can include an outlet (25) for products produced in the reaction zone (28). In preferred aspects, the majority of the products produced is benzene. However, other products can include acetaldehyde, crotonaldehyde, butene, furan, and ethylene. The collection device (23) can be in fluid communication with the reactor (22) via the outlet (25). Both the inlet (27) and the outlet (25) can be open and closed as desired. The collection device (23) can be configured to store, further process, or transfer desired reaction products (e.g., benzene) for other uses. By way of example only, FIG. 1 provides non-limiting uses of benzene produced from the catalysts and processes of the present invention. Still further, the system (20) can also include a heating source (26). The heating source (26) can be configured to heat the reaction zone (28) to a temperature sufficient (e.g., 350 K to 700 K) to convert ethanol in the ethanol feed to benzene. A non-limiting example of a heating source (26) can be a temperature controlled furnace.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### Catalyst Preparation and Characterization

TiO₂ anatase nanoparticles and TiO₂ rutile nanofibers were prepared by the sol-gel hydrolysis of Ti(IV) isopropoxide, and TiO₂-supported gold nano-particle catalysts (Au loading=1, 2, 4, and 8 wt. %) were prepared by the deposition-precipitation method with urea, as described in Jovic et al. (2013). TiO₂-sol was prepared by dissolving Ti(IV) iso-propoxide (284.4 g) in iso-propanol (1 L) at 293 K. Under vigorous stirring, ultrapure water (1 L) was added slowly drop wise to Ti(IV) iso-propoxide solution resulting in the hydrolysis of the alkoxide and precipitation of hydrous titanium oxides. The final molar ratio of water:Ti(IV) iso-propoxide in the reaction mixture was 55.5:1. The suspension was then left stirring for 24 hours. The particles were subsequently collected by vacuum filtration, washed repeatedly with isopropanol, and then air dried for two days at 293 K. Anatase nanoparticles were obtained upon calcination of the dried powders at 673 K for two hours.

TiO₂ inverse opal catalysts were prepared by the methods described in Waterhouse et al. (2008). TiO₂ inverse opals were prepared by filling the voids in polymethylmethacrylate (PMMA) colloidal crystal templates with a TiO₂-sol solution, followed by calcination to remove the PMMA template. TiO₂-sol was prepared by the same method used for TiO₂ nanoparticle preparation. TiO₂-sol was applied drop wise on PMMA colloidal crystals (5.0 g) deposited in thin layer over filter paper under strong vacuum applied to the Buchner funnel. The resulting TiO₂/PMMA composites were then washed repeatedly with isopropanol and air dried at 20° C. for two days. TiO₂ inverse opal powder was obtained upon calcination of the dried composite at 400° C. for 2 h.

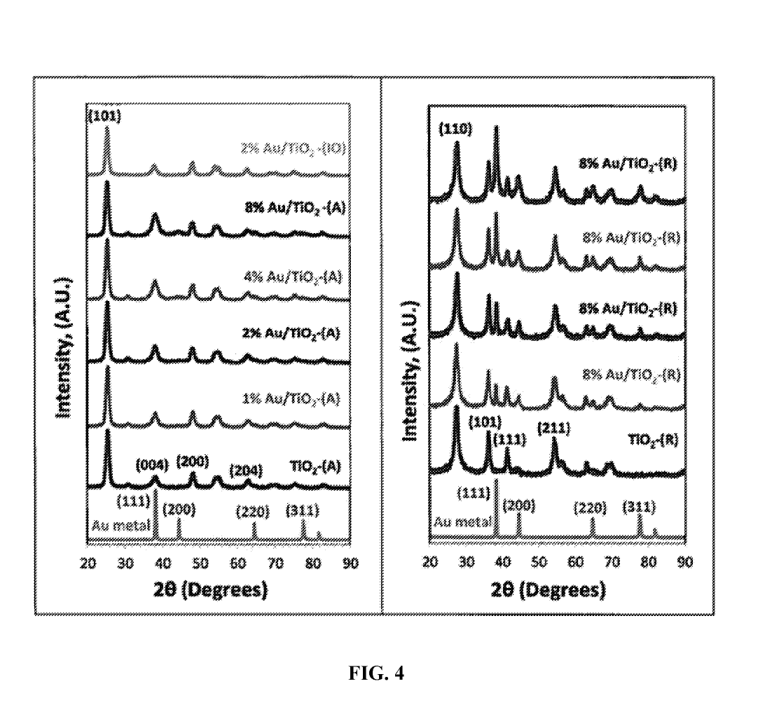

The Au/TiO₂ catalysts were characterized by BET, XRD, XPS, and TEM. The BET surface areas for all catalysts after supporting Au did not deviate from that of TiO₂ anatase nanoparticles (105 m² g_{Catal}⁻¹), rutile nanoparticles (170 m² g_{Catal}⁻¹), and TiO₂ inverse opal (60 m² g_{Catal}⁻¹) alone. The cumulative pore volume 0.26 cm³ g⁻¹ and average pore radius 4.0 nm typical for the anatase TiO₂ support were also unchanged. XRD revealed the broad features typical for nanocrystalline and nanofibrous TiO₂ as well as for Au particles as shown in FIG. 4.

As shown in FIG. 4, the XRD pattern of the micron size Au particles can be used as a reference to monitor the Au on TiO₂. In case of anatase catalysts the Au (111) reflection was not informative due to its overlapping with TiO₂ anatase (0004) reflection. Au (200) reflection was barely visible in the form of a broad peak in the 8 wt. % Au loading indicating the presence of very small Au particles. However, in the case of TiO₂ rutile catalysts Au (111) reflection was very clearly observed whose intensity increased with an increase in Au loading indicating the presence of large Au particles as compared to particles present on TiO₂ anatase. These results are in line with the results obtained by TEM studies discussed below.

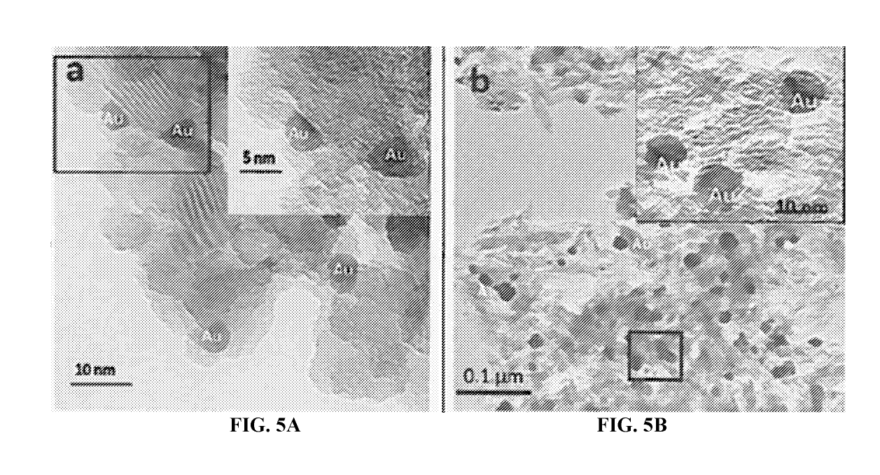

FIG. 5(A) illustrates images of 8 wt. % Au/TiO₂ anatase catalyst. Most gold particles are of similar sizes, less than ca. 10 nm. The deposition with the urea preparation method produces small gold particles in good contact with the TiO₂ anatase support, even at high Au loadings. FIG. 2(B) illustrates TEM images of the 8 wt. % Au/TiO₂ rutile nanofibers. Au particles have a wide distribution range of size (ca. 15-45 nm). The image at the top right corner of FIGS. 5(A) and (B) indicate that Au particles are in good contact with the support.

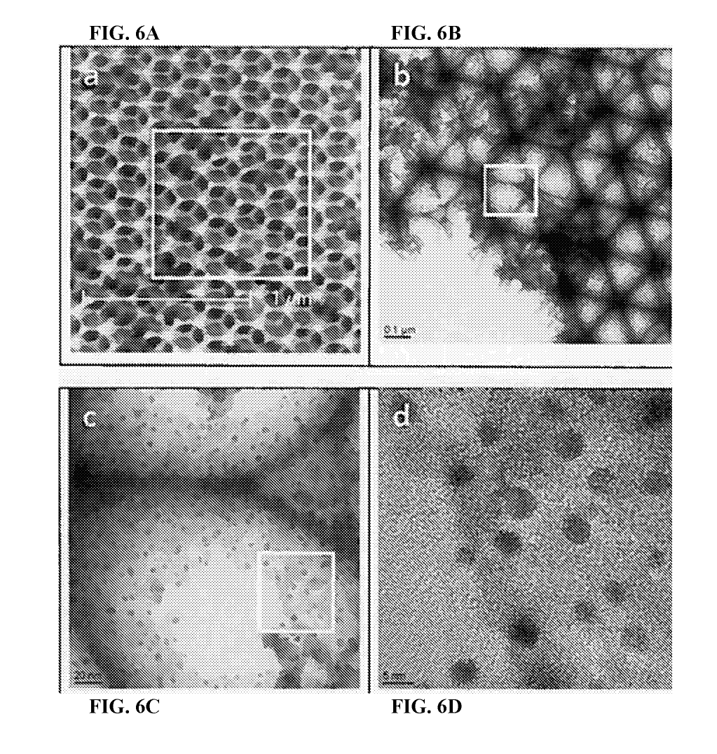

The SEM image (FIG. 6A) and TEM images (FIGS. 6B to 6D) of the 2 wt. % Au/TiO₂ inverse opal catalyst demonstrate the highly porous support phase and the small well-dispersed Au particles. As seen in FIG. 6A, TiO₂ inverse opal is a highly ordered, three-dimensional macroporous (3DOM) structure. The region inside the rectangle of FIG. 6A is shown in FIG. 6B. In FIG. 6B, the dark lines of the TEM image correspond to the walls of the macropores and the bright areas correspond to the windows formed due the presence of walls of the macropores underneath. The diameter of the macropores is around 215 nm, indicated by both the SEM and TEM images. The region inside the rectangle of FIG. 6B is shown in FIG. 6C. In FIG. 6C, the structure of the macropore wall can be observed and indicates that TiO₂ anatase nanoparticles (8-12 nm) form the walls of the macropores with Au particles supported on the surface thereof. There is present a second type of pores among the TiO2 particles within the walls called mesopores (10-15 nm). In FIG. 6D, the size of the Au particles is shown to be about 2-3 nm, and the Au particles are shown to be in good contact with the support.

### Example 2

### Ethanol Reactions

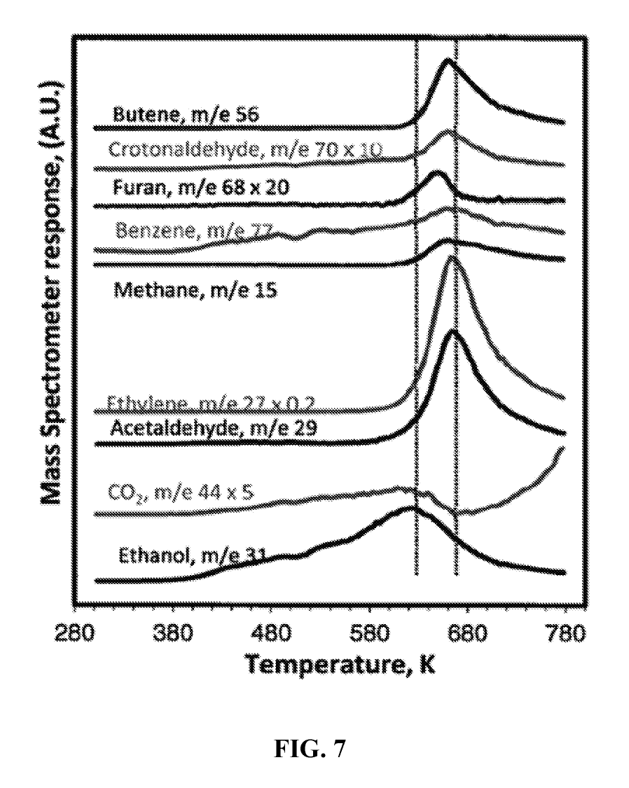

TPD products profile following ethanol adsorption at 300 K on H_{z}-treated TiO₂ is shown in FIG. 7. Based on the catalyst surface area (107 m²g⁻¹_{Catal.}) and number density of 5-fold coordinated Ti atoms on a TiO₂ surface (2 Ti atoms per 38.76 Å²=5.16×10¹⁸ Ti atoms per 1 m²), the number of Ti atoms available on the surface in 50 mg of TiO₂ loaded in the TPD reactor was approximately ca. 3×10¹⁹ atoms. Given the amount of ethanol used was 1 μL (1.03×10¹⁹ molecules) and assuming that all the ethanol molecules of the 1 μL of ethanol are adsorbed, the coverage is ca. 0.4. The reactivity of aliphatic alcohols with TiO₂ powders has been studied by using a number of different techniques and pre-treatments, including temperature programmed desorption spectroscopy (See, Rizzi et al. Physical Chemistry Chemical Physics. 1(4):709-11, 1999; Lusvardi et al. The Journal of Physical Chemistry. 100(46):18183-18191, 1996; Gamble et al., Surface Science. 348(1-2):1-16, 1996, and Lusvardi et al. Lusvardi et al., Journal of Catalysis. 153(1):41-53, 1995). In general, adsorption is largely dissociative yielding alkoxides and surface hydroxyl groups. In the present work ethanol is seen to desorb in the temperature domain 380 K to 700 K and accounted for 3.8% of the total product desorbed. Ethanol desorption profile can be deconvoluted to two peaks; a small one at about 460 K followed by a large desorption peak at about 620 K. The large peak might be attributed to ethoxide and hydroxyl recombination on surface oxygen defects. The most pronounced desorption signal is that of ethylene at 665 K contributing to 71.7% of the total product desorbed.

Assuming that surface coverage was initially about 0.4, then the number of sites involved in ethoxide conversion to ethylene was about 0.3. Ethylene is formed by ethoxide dehydration which can be linked to ethoxides adsorbed on oxygen defected sites. Because the number of oxygen defects sites prior to adsorption cannot reasonably exceed 30%, the dehydration reaction can be due in part to additional defects created during TPD. These defects can be created by the removal of surface water as follows:

CH₃CH₂OH_{(g)}+Ti₍ₛ₎O₍ₛ₎→CH₃CH₂OTi₍ₛ₎+OH₍ₐ₎ (1)

2OH₍ₐ₎→H₂O_{(g)}+Vₒ (2)
where: (a) for absorbed; Vₒ for surface oxygen vacancy; (s) for surface; and (g) for gaseous phase.

It is clear from equations (1) and (2) above that the number of Vₒ is at maximum ½ of the number of ethoxides/ethanol(a) and therefore can, at maximum, be equal to a surface coverage of 0.2. Therefore, the formation of ethylene can be linked to a combined number of surface oxygen defects created prior to adsorption and those formed during TPD amount other factors.

Table 1 provides the carbon % yield and selectivity from ethanol-TDP on TiO₂ nanoparticles, after overnight reduction at 723 K with H₂.

TABLE 1

| | | Carbon | Carbon |
|---|---|---|---|
| Product | Peak Temperature (K) | Yield (%) | Selectivity (%) |
| Ethanol | (380-550, 615) | 0.2, 3.6 | — |
| Acetaldehyde | 660 | 4.0 | 4.2 |
| Ethylene | 660 | 71.7 | 74.5 |
| Butene | 660 | 9.6 | 10.0 |
| Methane | 660 | 1.8 | 1.9 |
| Crotonaldehyde | 385-605, 665 | 0.02, 0.4 | 0.02, 0.42 |
| CO₂ | 610 | 0.1 | 0.1 |
| Benzene | 365-580, 650 | 0.6, 7.9 | 0.6, 8.2 |
| Furan | 385-580, 650 | 0.02, 0.1 | 0.02, 0.1 |
| Total | LT, HT* | 0.84, 95.5 | 0.64, 99.3 |

*LT and HT indicate total carbon % yield at low temperature (LT) and high temperature (HT), respectively. The carbon yield involves the corrected peak area of a desorbing product times its number of carbon. The following equation is illustrative:
${Y}_{i} = \frac{{PA}_{i} \times {CF}_{i} \times {Cn}_{i}}{{\sum }_{j}^{n} {PA}_{j} \times {CF}_{j} \times {Cn}_{j}} ,$

where PAᵢ is the area under the peak, CFᵢ is the correction factor, Cnⱼ is the number of carbon atoms in the molecule. j is for all products including i. The carbon selectivity is the same taking away the reactant (ethanol in this case).

A smaller fraction of ethoxides gave acetaldehyde by dehydrogenation (about 4%). It has been seen numerous times that the dehydrogenation reaction is far weaker on TiO₂ compared to the dehydration reaction. One can view this reaction as due to the removal of a hydride from the ethoxide as follows:

CH₃CH₂O(a)+OH(a)→CH₃CHO(g)+H₂(g)+O(s) (3)

It is important to emphasize that the hydrogen removed from the ethoxide is a hydride (H^{δ-}, i.e. a negatively charged H) that recombines with the hydrogen ion (H^{δ+}) if the hydroxide to form H₂. The amount of acetaldehyde desorbed is not a true indicator of the extent of the dehydrogenation reaction on a reduced TiO₂ surface. There are other reactions which compete with acetaldehyde desorption. The most important of these reactions is the reductive coupling of carbonyl compounds to olefins known as McMurray reaction and has not been observed previously on reduced TiO₂ single crystals. In this study, TPD results on TiO₂ alone (FIG. 7 and Table 1) indicated considerable desorption of butane (9.6%) that is formed through this reaction:

2CH₃CHO+2Vₒ→CH₃CH═CHCH₃+2O₍ₛ₎ (4)

Together with butane desorption there is also a small desorption of crotonaldehyde (0.4%) via β-aldolisation (condensation of two acetaldehyde molecules followed by dehydration). Therefore, the true activity of TiO₂ to acetaldehyde is close to 20% (taking into account reaction stoichiometry of the formation of butane and crotonaldehyde). In other words, the dehydration to dehydration ratio is around 3. Notably, there is a non-negligible amount of benzene formation. While benzene production has previously been seen from ethanol during TPD on other surfaces (see Idriss et al. (1996); Wu et al. (2009); Yee et al. (2000)), but has not yet been reported on H₂-reduced TiO₂. This reaction is discussed in further detail below with respect to Au/TiO₂ catalyst as the addition of Au increases benzene formation considerably. A small amount of methane with overall carbon selectivity of 1.9% desorbed at high temperature. CO₂ was seen to desorb in trace quantities at 610 K.

Additionally, it is worth noting that the overall reaction yields 3 moles of hydrogen per mole of benzene in its stoichiometric form.

3CH₃CH₂OH→C₆H₆+3H₂O+3H₂

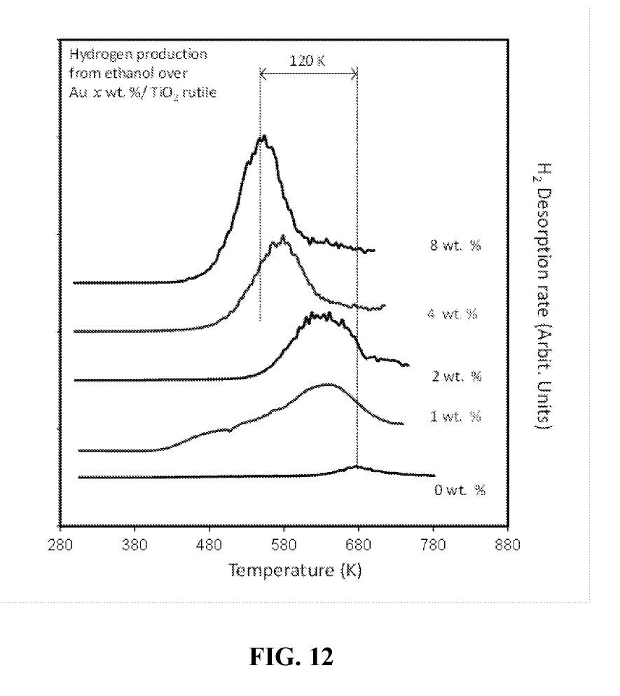

To illustrate this FIG. 12 presents Ethanol-TPD results where hydrogen is produced using different Au/TiO₂ rutile catalysts. The experiments are similar if TiO₂ is in the form of Anatase or if the catalysts were prior reduced with hydrogen. FIG. 12 shows the increasing production of hydrogen with increasing amounts of the metal (Au) mirroring the production of benzene.

### Example 3

### Au/TiO₂₋ₓ Nanoparticles

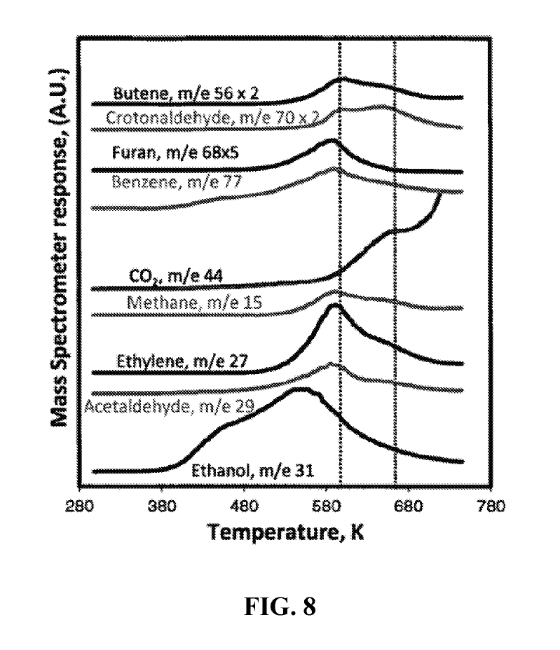

In this work, the effect of Au loading on ethanol TPD reaction products was conducted for a series of H₂-treated Au/TiO₂ (Anatase) nanoparticle catalysts with 1, 2, 4, and 8 wt. % Au loading. In general, Au loading affected the TPD desorption products temperature and distribution gradually. However, the effect of added Au was most pronounced on 8 wt. % Au/TiO₂ catalyst and is presented in more details. The TPD product profile following ethanol adsorption at 300 K on this catalyst is shown in FIG. 8. Unreacted ethanol (m/z 31) started to desorb at 380 K very similar to pure TiO₂ nanoparticles, however, conversion to other products is seen about 60 K lower than in case of TiO₂ alone. The overall desorption of unreacted ethanol is found to be 12% of the total carbon yield. Benzene was also desorbed at 380 K contributing to 10.4% carbon yield at this temperature range and 50.3 at 585 K with other desorption products at higher temperature domain. Aside from benzene and ethanol, no other produces were seen to desorb in the 380 K temperature domain.

The majority of the products desorbed at temperatures above 580 K in two desorption domains. In the case of pure TiO₂, only one desorption domain was observed. It can be noticed that Au loading shifts the product desorption in the high temperature domain towards lower temperature. However, there are still some desorption occurring due to TiO₂ sites no in proximity of Au. Therefore, it appears that ethoxide species close to Au particles react differently than those on TiO₂ alone. The carbon selectivity for products desorbing at 590 K was found equal to 76.6% while that at 640 K is equal to 11.7%. Carbon yields and carbon selectivities at the individual desorption temperatures are summarized in Table 2. It can be noticed that for the Au/TiO₂ catalyst, benzene is the most dominant desorption product with a total carbon selectivity of 69.1%, most of which desorbed at 590 K along with ethylene (dehydration), acetaldehyde (dehydrogenation), and other minor products including butane, crotonaldehyde, and furan. Very small amounts of methane with carbon selectivity of 0.5% were also detected. CO₂ was detected only at the highest temperature of 650 K.

TABLE 2
(Carbon % yield and selectivity from ethanol-TPD on
Au/TiO₂ nanoparticles, after overnight reduction at 723 K with H₂)

| | Peak | | Carbon | |
|---|---|---|---|---|
| | Temperature | Carbon | Selectivity | Ratio |
| Product | (K) | Yield (%) | (%) | MT/HT |
| Ethanol | 355-500, 540 | 3.9, 8.3 | — | — |
| Acetaldehyde | 585, 640 | 3.9, 1.6 | 4.4, 1.8 | 2.4/1 |
| Ethylene | 590, 640 | 7.0, 4.2 | 8.0, 4.8 | 17/1 |
| Butene | 600, 640 | 2.0, 1.7 | 2.3, 2.0 | 1.2/1 |
| Methane | 590, 640 | 0.5, 0.5 | 0.5, 0.5 | 1/1 |
| Crotonaldehyde | 600, 640 | 1.3, 1.8 | 1.5, 2.0 | 0.7/1 |
| CO₂ | 650 | 0.5 | 0.6 | — |
| Benzene | 355-477, 585 | 10.4, 50.3 | 11.8, 57.3 | 50/0 |
| Furan | 585 | 2.3 | 2.6 | 2.3/0 |
| Total | LT, MT, | 14.3, 76, 9.8 | 11.8, 76.6, 11.7 | 6.5/1 |
| | HT* | | | |

*LT, MT, and HT indicate total carbon % yield at low, middle, and high temperatures, respectively.

### Example 4

### Benzene Formation

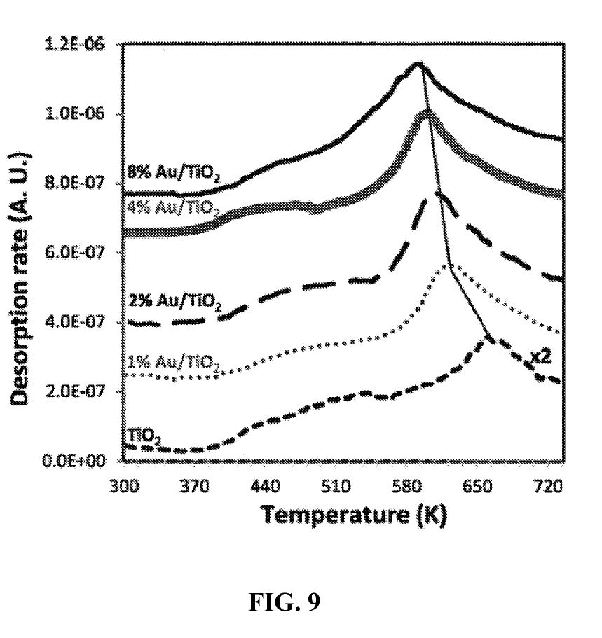

On TiO₂ anatase ethylene was the major product desorbed with overall carbon selectivity of 74% while on Au/TiO₂ benzene selectivity increased with an increase in Au loading and was the major product with ˜70% carbon selectivity on 8 wt. % Au/TiO₂. FIG. 9 shows the effect of Au loading on benzene formation in the case of H₂-reduced catalysts where the decrease in the benzene desorption temperature and an increase in its amount with increase in Au loading can be observed clearly. A possible explanation for the higher benzene selectivity on Au/TiO₂ might be that ethylene is converted to benzene by a trimerization/dehydrogenation-type reaction. This explanation is most likely not accurate. In particular, previous work has shown that the formation of crotonaldehyde from acetaldehyde over powder CeO₂ (Idriss et al. Journal of Catalysis. 155(2):219-237, 1995), reduced single crystal UO₂(111) (Chong et al. Journal of Vacuum Science & Technology A. 19:19333-1937, 2001), powder UO₂, U₃O₈, Al₂O₃ (Madhavaram et al. Journal of Catalysis 224:358-369, 2004). Crotonaldehyde formation has also been observed, from acetaldehyde, over TiO₂ single crystals as well as powders (Idriss et al. Journal of Catalysis. 139(1):119-133, 1993). Its formation requires both coordinated unsaturated Ti cations to act as Lewis acid sites to bind acetaldehyde and a nearby basic site (oxygen anion) to abstract an α-H from acetaldehyde. The abstraction of a proton from the α-position of acetaldehyde by lattice oxygen results in the formation of a —CH₂CHO(absorbed) and a surface hydroxyl group. The former is a nucleophilic species which can react with the electrophilic carbonyl group of second acetaldehyde molecule adsorbed on an adjacent Ti cation to give an adsorbed aldol. The aldol thus formed further dehydrates to crotonaldehyde. However, the amount of crotonaldehyde desorbed during TPD is small over the Au/TiO₂ catalyst. This can be explained as follows. Once crotonaldehyde is formed, it can react with another adsorbed acetaldehyde (via the same β-aldolisation reaction) giving 2,4-hexadienal (see equation 5 above). On contact with Au, it may undergo C—H bond scission of the methyl group which after intramolecular cyclisation followed by H₂0 elimination may give benzene as shown in reaction scheme 1:

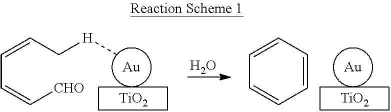

The TiO₂ nanoparticles used in this work have high surface area (more adsorption sites) and small pore size (˜4 nm in size). This provides not only more active sites for re-adsorption but also hinders the diffusion of bulky molecules like 2,4-hexadienal.

### Example 5

### Dehydration Vs. Dehydrogenation

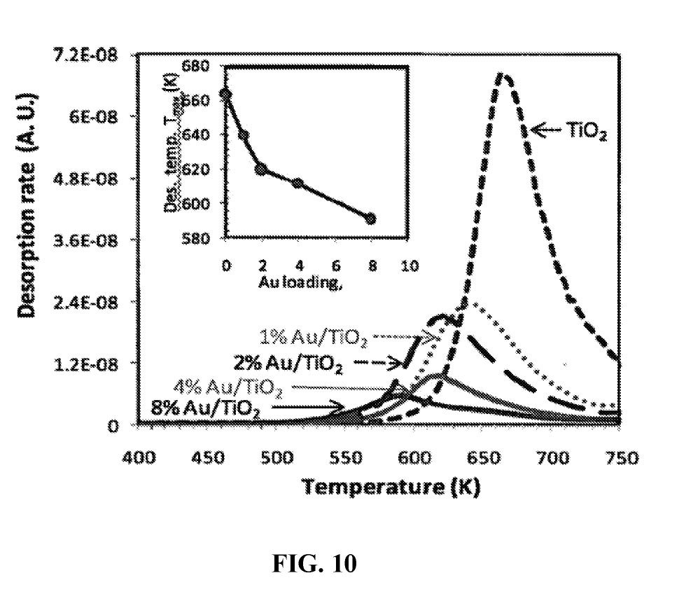

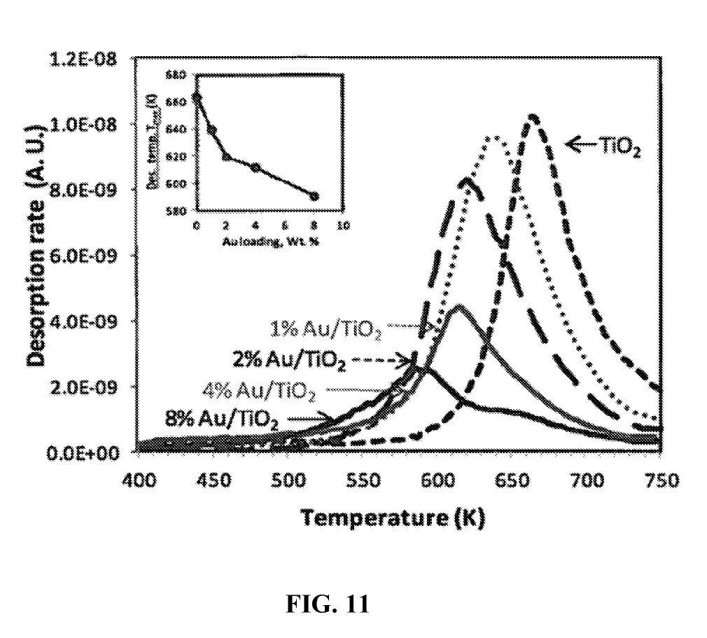

FIGS. 10 and 11 indicate the desorption profiles of ethylene and acetaldehyde from Au/TiO₂ catalysts at indicated Au loadings as a function of temperature. The data in these FIGS. 10 and 11 confirm: (1) There is a shift of both ethylene and acetaldehyde towards lower temperature with increase in Au loading; (2) There is an abrupt drop in rate of ethylene desorption from pure TiO₂ to Au supported TiO₂. However, there is a gradual decrease in the rate of ethylene desorption with increasing Au loading in the case of Au supported catalysts; and (3) In contrast to the decrease in ethylene desorption rate, the decrease in acetaldehyde acetaldehyde desorption rate is relatively slow which leads to an increase in the acetaldehyde to ethylene ratio with increased Au loading.

The insets in both FIGS. 10 and 11 represent the temperature at which the rate of both ethylene and acetaldehyde desorption is maximum as a function of Au loading. Lowering in desorption temperature is similar for both products with a maximum lowering of up to about 60 K in the case of 8 wt. % Au/TiO₂ as compared to pure TiO₂. This indicates that the activation energy for dehydration and dehydrogenation is lowered with increase in Au loading. Acidic oxides such as Al₂O₃ are found to make considerable amounts of ethylene while on the other hand, basic oxides such as CeO₂ have the opposite effect. TiO₂ also gives high yield of ethylene. In this case it has been seen that ethylene desorption rate is suppressed with increased Au loading. As dehydration reaction is believed to occur at defect sites, it is reasonable to assume that Au affect these sites.

These data confirm that the presence of Au particles in the case of Au/TiO₂ anatase had two noticeable effects: (1) It decreases the overall desorption at high temperature by up to 60 K; and (2) It shifts the reaction selectivity from ethylene (dehydration) to acetaldehyde (dehydrogenation), the latter further reacts by condensation reactions leading ultimately to benzene. This likely means that the abstraction of the H atom in the alpha position from the C—O (as a hydride) is favored in the presence of Au compared to that of the H atom in the beta position of the C—O (as a proton) in the absence of Au.

1. A catalyst capable of producing benzene from ethanol comprising:
a titanium dioxide support;
gold nanostructures dispersed on the surface of the titanium dioxide support; and
ethanol adsorbed onto the surface of the titanium dioxide support;
wherein the catalyst is capable of producing benzene and hydrogen from the adsorbed ethanol such that the benzene carbon yield from the adsorbed ethanol is at least 10% when the catalyst is heated to a temperature of 350 to 700 K.

2. The catalyst of claim 1, wherein the catalyst comprises 1 to 10 wt. % of the gold nanostructures.

3. The catalyst of claim 1, wherein the titanium dioxide support comprises titanium dioxide nanostructures or microstructures.

4. The catalyst of claim 3, wherein the titanium dioxide nanostructures include nanoparticles or nano-fibers or a combination thereof.

5. The catalyst of claim 4, wherein the nanoparticles have an average size of 10 to 20 nm and the nano-fibers have an average width of 10 to 30 nm and an average length of 40 to 60 nm.

6. The catalyst of claim 1, wherein the titanium dioxide support has an inverse opal structure, wherein the inverse opal structure has pores having an average size of 175 to 400 nm.

7. The catalyst of claim 1, wherein the titanium dioxide is reduced titanium dioxide.

8. The catalyst of claim 1, wherein the titanium dioxide has an average pore size of less than 10 nm.

9. The catalyst of claim 1, wherein the gold nanostructures are nanoparticles having an average size of less than 15 nm, less than 10 nm, or less than 5 nm.

10. The catalyst of claim 1, wherein the catalyst is capable of producing benzene from the adsorbed ethanol such that the benzene carbon yield from ethanol is at least 20% when the catalyst is heated to a temperature of 500 to 700 K.

11. The catalyst of claim 1, wherein the catalyst is capable of producing benzene from the adsorbed ethanol such that the benzene carbon yield from ethanol is 10 to 70% when the catalyst is heated to a temperature of 500 to 700 K.

12. The catalyst of claim 11, wherein the temperature is 550 to 650 K or 550 to 600 K or about 585 K and the majority of carbon from the adsorbed ethanol is present in the produced benzene.

13. A method of producing benzene from the catalyst of claim 1, the method comprising heating the catalyst to a temperature of 350 to 700 K, wherein benzene is produced such that the benzene carbon yield from ethanol is at least 10%.

14. The method of claim 13, wherein the benzene carbon yield from ethanol is at least 10% up to 70%.

15. The method of claim 14, wherein benzene is absorbed onto the surface of the catalyst, and wherein the benzene carbon yield from the adsorbed ethanol is 5 to 15% at a temperature of 350 to 400 K and 40 to 60% at a temperature of 550 to 650 K.

16. The method of claim 15, wherein benzene is produced and desorbed at the interface of the titanium dioxide support and the gold nanostructures.

17. The method of claim 16, wherein the benzene is formed by cyclization of a hexadienal intermediate.

18. A method of producing the catalyst of claim 1, the method comprising:
(a) dispersing gold nanostructures onto at least a portion of a surface of a titanium dioxide support to produce titanium dioxide supported gold nanostructures;
(b) contacting the titanium dioxide supported gold nanostructures with ethanol under conditions sufficient to adsorb ethanol onto the surface of the titanium dioxide; and
(c) obtaining the catalyst.

19. The method of claim 18, wherein the titanium dioxide supported gold nanostructures from step (a) are reduced with a reducing agent prior to step (b).

20. The method of claim 18, wherein step (b) is performed at a temperature of 150° C. to 500° C.
